(19) United States
(12) Patent Application Publication
(10) Pub. No.: US 2006/0264707 A1
(43) Pub. Date: Nov. 23, 2006

(54) Endoscope sheath

(76) Inventor: Timothy P. Kinney, Chicago, IL (US)

Correspondence Address: WESTMAN CHAMPLIN & KELLY, P.A., SUITE 1400, 900 SECOND AVENUE SOUTH, MINNEAPOLIS, MN, 55402-3319, US

(21) Appl. No.: 11/133,550

(22) Filed: May 20, 2005

(51) Int. Cl. A61B 1/00

(52) U.S. Cl. 600/115; 600/116; 600/156

(57) ABSTRACT

A sheath and a method of operating the sheath are provided for assisting movement of an endoscope within a cavity of a patient's body. The sheath includes an everting shaft having an internal and external lining with an inflatable lumen between them, proximal and distal regions and a lumen for receiving the endoscope. An expandable member is positioned along the distal region, which is expandable from a first diameter, at which the expandable member grips the endoscope to a second, larger diameter at which the expandable member engages a wall of the cavity.

### FIELD OF THE INVENTION

The present invention relates to medical devices and more particularly to endoscopes for performing colonoscopy and other medical procedures.

### BACKGROUND OF THE INVENTION

Flexible colonoscopy has been performed for more than 30 years. While significant advances have been made during that time, the procedure is still relatively unpleasant for most patients, and quite painful for others requiring not insignificant amounts of pain sedation. During the procedure, the physician advances a flexible endoscope through the soft and winding colon. During advancement, the physician pushes one end of the endoscope while trying to direct the distal tip (that can be up to four feet away from the physician) through the colon. Since the endoscope is flexible, the force applied to the endoscope at the proximal end is not necessarily transferred to the distal end. Rather, the endoscope tends to form bends and loops as it is being pushed through the colon.

This inevitably leads to stretching of the colon and of the various points where the colon is tethered inside the body causing discomfort for the patient. As a result, physicians typically need to administer sedation to the patient in order for the patient to remain comfortable through the procedure and reduce the pain created by the stretch. Sedation carries significant risks, particularly when higher doses are used, including depressing the respiratory and cardiac function.

Recent technical advances in colonoscopies over the past several years include thinner endoscopes, better optics and adjustable stiffness of the scope shaft, which assists in advancing the scope more easily into the colon. However, the basic technique is unchanged and problems of overstretching the colon remain. The procedure still remains unpleasant for the patient and most often requires considerable sedation.

Improved endoscopes and colonoscopy procedures are therefore desired.

### SUMMARY OF THE INVENTION

One embodiment of the present invention is directed to a sheath for assisting movement of an endoscope within a cavity of a patient's body. The sheath includes an everting shaft having proximal and distal regions and a lumen for receiving the endoscope. An expandable member is positioned along the distal region, which is expandable from a first diameter to a second, larger diameter for engaging a wall of the cavity.

Another embodiment of the present invention is directed to an endoscope apparatus. The apparatus includes an everting shaft having inner and outer layers and a lumen defined by the inner layer. An endoscope extends along the lumen and is engaged with the inner layer. An expandable member is positioned along a distal region of the outer layer and is expandable from a first diameter at which the expandable member grips the endoscope to a second, larger diameter at which the expandable member releases the endoscope and allows the endoscope to move relative to the expandable member.

Another embodiment of the present invention is directed to a method of moving an endoscope along an elongated body cavity. The method includes: (a) inserting an endoscope apparatus into the body cavity, the apparatus comprising an endoscope and an everting shaft, which includes inner and outer layers, a lumen through which the endoscope extends, and an expandable member; (b) advancing the apparatus to a curved portion of the body cavity with the expandable member contracted to a first diameter at which the expandable member grips the endoscope; (c) expanding the expandable member from the first diameter toward a second, larger diameter at which the expandable member releases the endoscope and anchors to a wall of the body cavity; (d) applying a withdrawing force on the outer layer when the expandable member is anchored to the wall of the body cavity to thereby reduce curvature of the curved portion of the body cavity; and (e) advancing the endoscope relative to the expandable member such that engagement between the endoscope and the inner layer causes a distal portion of the inner layer to evert to the outer layer during advancement.

Another embodiment of the present invention is directed to a medical device for insertion into a body cavity. The device includes an elongated sheath. The expandable member is expandable from a first diameter to a second, larger diameter and is mounted to the elongated sheath such that the elongated sheath has an angular position that is rotatable relative to the expandable member.

### BRIEF DESCRIPTION OF THE DRAWINGS

FIGS. 1-1 through 1-14 illustrate a colonoscopy procedure according to one embodiment of the present invention.

FIG. 2 is a side view of an endoscope apparatus in a deflated state according to one embodiment of the present invention.

FIG. 3 is a side view of the endoscope apparatus in an inflated state according to one embodiment of the present invention.

FIG. 4 is an enlarged, cross-sectional view of the distal end of the apparatus shown in FIGS. 2-3 within a patient's colon.

FIG. 5 is an enlarged view of the distal end showing an expandable member in an inflated state.

FIG. 6 is an enlarged view of the distal end showing the expandable member in a deflated state.

FIG. 7 is a side view of an endoscope apparatus according to an alternative embodiment of the present invention.

FIG. 8 is a cross-sectional view of an endoscope apparatus having the inflation cavity of an expandable member coupled to the inflation cavity of an everting sheath according to an alternative embodiment of the present invention.

FIG. 9 is a cross-sectional view of an endoscope apparatus having a rotatable balloon according to an alternative embodiment of the present invention.

### DETAILED DESCRIPTION OF THE PREFERRED EMBODIMENTS

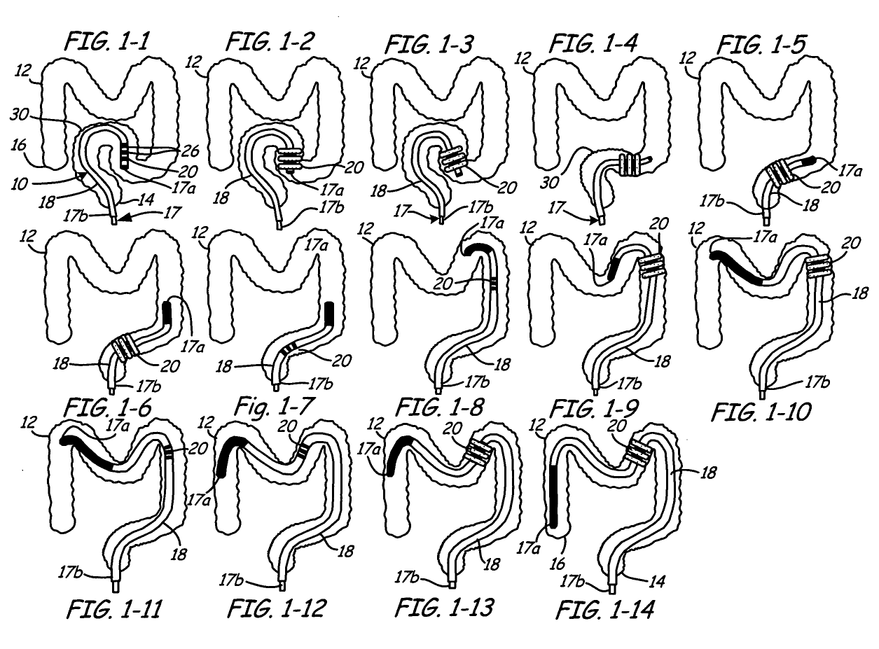

FIGS. 1-1 through 1-14 illustrate a colonoscopy procedure according to one embodiment of the present invention. During the procedure, an endoscope apparatus 10 is advanced into the colon 12 of a patient. The endoscope apparatus 10 is inserted into the bottom 14 of colon 12 and advanced through each segment to reach the end 16. For simplicity, the full length of endoscope apparatus 10 is not shown beyond the bottom 14 of colon 12 in FIGS. 1-1 through 1-14.

As described in more detail below, apparatus 10 includes an endoscope 17, which extends through an outer everting sheath 18 and has a distal end 17a and a proximal portion 17b. Sheath 18 assists in advancing endoscope 17 through colon 12. In one embodiment, everting sheath 18 has an inner layer that frictionally engages the outer diameter surface of endoscope 17 and an outer layer that carries an expandable member 20. In one embodiment, expandable member 20 forms a balloon, which can be inflated and deflated. When sheath 18 and expandable member 20 are inflated, the outer layer of the sheath becomes separated from the inner layer. The inner layer is connected to the outer layer at the distal end of sheath 18 to allow the inner layer to evert to the outer layer at the distal end of sheath 18.

In FIG. 1-1, the endoscope apparatus 10 is shown inserted into colon 12, within the everting sheath 18, which is fully deflated. When deflated, one or more elastic elements or bands 26, grip the expandable member 20 and sheath 18 to the outer diameter of endoscope 17 such that the endoscope and sheath can be advanced together along colon 12.

At this point in the procedure, the endoscope is used in a normal manner. As the endoscope is advanced further within the colon, the shaft of the endoscope begins to form bends and loops, causing discomfort due to stretching of the colon from its normal configuration. This is usually the first point in the procedure when the patient experiences significant discomfort. As the physician pushes proximal portion 17b in order to advance distal end 17a, the bend in endoscope 17 can cause the colon 12 to stretch along area 30 causing pain.

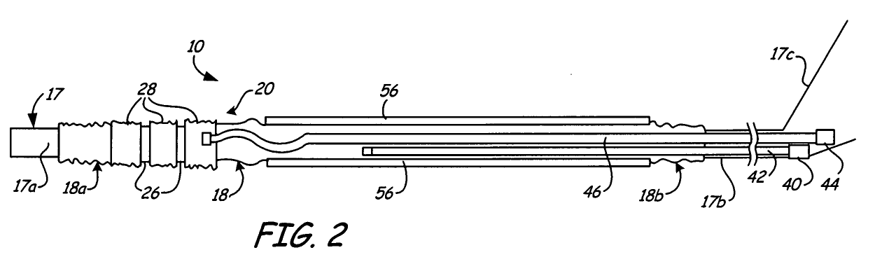

FIG. 1-2 illustrates everting sheath 18 and expandable member 20 inflated with a fluid such as water. However, any suitable fluid (liquid or gas) can be used for inflating sheath 18 and/or expandable member 20. Sheath 18 and expandable member 20 can be inflated through a common passage or lumen or can be inflated through separate passages or lumens. When expandable member 20 and sheath are inflated, the water pressure overcomes the grip of the small elastic bands 26 (shown in FIG. 1-1) such that expandable member 20 and the outer layer of sheath 18 no longer grip the outer diameter of endoscope 17. Expandable member 20 is sized and shaped such that, when inflated, the outer diameter surface of expandable member 20 engages the interior surface of colon 12 and can serve to anchor sheath 18 in position relative to the colon. Various shapes and textures of the expandable member can be used to anchor the sheath and expandable member to the colon without applying excessive radial force on the colon, as excessive radial force could cause discomfort in and of itself. When the sheath and expandable member are anchored in position, endoscope 17 is free to move in an axial direction relative to expandable member 20.

In FIG. 1-3, with expandable member 20 anchored to colon 12, the physician pulls endoscope 17 and sheath 18 from proximal portion 17b to withdraw slightly the endoscope and sheath from the colon and thereby straighten that section of the colon.

In FIG. 1-4, the bottom of colon 12 begins to shorten and the stretch along area 30 is reduced as endoscope 17 and sheath 18 are withdrawn further. Eventually, the curve formed by endoscope 17 is reduced with further withdrawal of endoscope 17 and sheath 18, creating a relatively straight section of colon 12 for advancement of the endoscope along the colon.

Referring to FIG. 1-5, expandable member 20 remains inflated and is fixed against the wall of colon 12. Endoscope 17 is able to move independently from expandable member 20 because of the action of everting sheath 18 underneath the expandable member. The physician can then push the proximal portion 17b of the endoscope while maintaining a pulling force on the outer layer of sheath 18. As shown in FIG. 1-6, the frictional engagement between the outer diameter surface of endoscope 17 with the inner layer of sheath 18 causes the inner layer of the sheath to evert out of the distal end of the sheath (as shown by everted section 32). As sheath 18 everts, more of the distal end 17a of the endoscope becomes exposed.

As shown and described in more detail below, everted section 32 represents material along the inner layer of sheath 18 that has advanced relative to the outer layer of the sheath and is therefore exposed out of the distal end of the sheath.

At this point in the procedure, the distal end 17a of endoscope 17 has reached a relatively straight section of colon 12 due to the everting action of sheath 18 and the straightening of the bottom portion of the colon. Expandable member 20 and sheath 18 can then be deflated, as shown in FIG. 1-7. This causes elastic bands 26 to re-grip endoscope 17 at a distance further back from distal end 17a. Endoscope 17 and sheath 18 can then be advanced together once again in the usual manner, as shown in FIG. 1-8.

When the patient starts to feel pain or the endoscope begins to form exaggerated bends once again, expandable member 20 can be re-inflated, as shown in FIG. 1-9. As shown in FIG. 1-10, the everting sheath 18 can be withdrawn slightly thereby straightening endoscope 17 between expandable member 20 (which is fixed against colon 12) and the rectum. This allows the endoscope 17 to be advanced in a relatively straight line between the rectum and expandable member 20. Now, most of the force applied by pushing endoscope 17 from proximal portion 17b is transferred to the level of expandable member 20. This has the effect of pushing the flexible endoscope 17 from the midshaft rather than at the proximal portion 17b, providing some mechanical advantage.

Once endoscope 17 has then been advanced beyond a difficult section and the colon is straightened, expandable member 20 can be deflated as shown in FIG. 1-11. Note that as sheath 18 becomes further everted, more of endoscope 17 becomes exposed out of the distal end of the sheath, along everted section 32. When expandable member 20 is deflated, the expandable member re-grips endoscope 17, this time even further back from distal end 17a. In FIG. 1-12, endoscope 17 and sheath 18 are advanced further until another difficult point is reached. In FIG. 1-13, expandable member 20 can be inflated once again. In FIG. 1-14, endoscope 17 is advanced to the end 16 of colon 12. At this point in the procedure, endoscope 17 can image the end 16 of colon 12.

If during the procedure the physician wishes to “reload” expandable member 20 to the distal end 17a of endoscope 17, the physician can advance endoscope 17 and deflated expandable member 20 to an straight section of colon 12 such that shown in FIG. 1-7, for example. The expandable member 20 can be inflated to engage the surface of colon 12 and hold the expandable member and sheath in a fixed position relative to the colon. The physician can then pull back and withdraw the endoscope 17 relative to sheath 18 and expandable member 20 until the distal end 17a of endoscope 17 reaches expandable member 20. Expandable member 20 and sheath 18 can then be deflated causing elastic bands 26 to re-grip the distal end 17a of endoscope 17. The endoscope 17, sheath 18 and expandable member 20 can then be advanced together through the straight section of colon 12.

The everting sheath and expandable member described above can be used with any type of endoscope for any medical procedure. In addition to endoscopes used for colonoscopy, the everting sheath and expandable member can be used with endoscopes for performing an upper endoscopy procedure through the esophagus, for example, to facilitate deeper passage into the small intestine. In addition, the sheath and expandable member can be used in other elongated medical instruments or devices for advancement along a human or other animal body cavity.

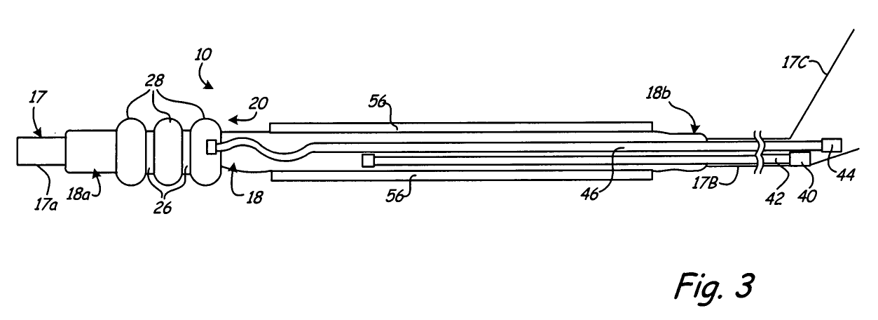

FIG. 2 is a side view of endoscope 17 and sheath 18, with sheath 18 and expandable member 20 in a deflated state according to one embodiment of the present invention. FIG. 3 is a side view of endoscope 17 and sheath 18, with sheath 18 and expandable member 20 in an inflated state according to one embodiment of the present invention.

Referring to FIG. 2, endoscope 17 has an elongated shaft with a distal end 17a, a proximal portion 17b and a handle 17c. Endoscope 17 is shown inserted within everting sheath 18. Everting sheath 18 has a distal end 18a and a proximal end 18b. Endoscope 17 extends through an internal lumen of sheath 18, from proximal end 18b to distal end 18a. In one embodiment, expandable member 20 is a separate element that is attached to the outer layer of sheath 18. In another embodiment, expandable member 20 is formed as a single, continuous piece of material with the outer layer of sheath 18. For example, expandable member 20 can be defined by one or more areas of reduced material thickness relative to the thickness of sheath 18, or by one or more areas where the sheath material elasticity is increased relative to the elasticity of sheath 18. The areas of reduced material thickness or increased material elasticity expand to a greater degree than sheath 18 when sheath 18 is inflated. Expandable member 20 can have any number of sections 28 defined by elastic bands 26.

Elastic bands 26 extend around the periphery of expandable member 20 and are sized to grip the outer diameter of endoscope 17 when sheath 18 and expandable member 20 are deflated. Similar to expandable member 20, elastic bands 26 can be formed of any suitable elastic material, which can be separate from expandable member 20 or integral with the expandable member material. For example, bands 26 can be separate rubber bands that are mounted over expandable member 20. In an alternative embodiment, elastic bands 26 are formed within the material of expandable member 20, such as one or more bands having increased material thickness than expandable member 20. Any number of bands can be used. Sheath 18, expandable member 20, and bands 26 can be formed of any suitable elastic material, such as elastic polymers.

Sheath 18 includes an inflation valve or port 40 for inflating sheath 18 through a lumen 42. In this embodiment, sheath 18 further includes a separate inflation valve or port 44 for inflating expandable member 20 through a separate lumen 46. Again, sheath 18 and expandable member 20 can be inflated through a common lumen or through separate lumens. As described in more detail below, sheath 18 is formed as a cylindrical tube with sidewalls formed by two layers of material with a space between them. When a fluid is introduced within the space between the two layers, the fluid pressure expands the space, thereby inflating the sheath, as shown in FIG. 3. The expandable member 20 is attached to the outer layer of material and can be inflated with sheath 18 or separately from sheath 18 in alternative embodiments.

Inflation valve 40 can include a fitting for attaching a syringe that can be used for inflating the sheath and/or expandable member. In addition, inflation valve 40 can include a chamber for storing extra material of sheath 18 that can be used during eversion.

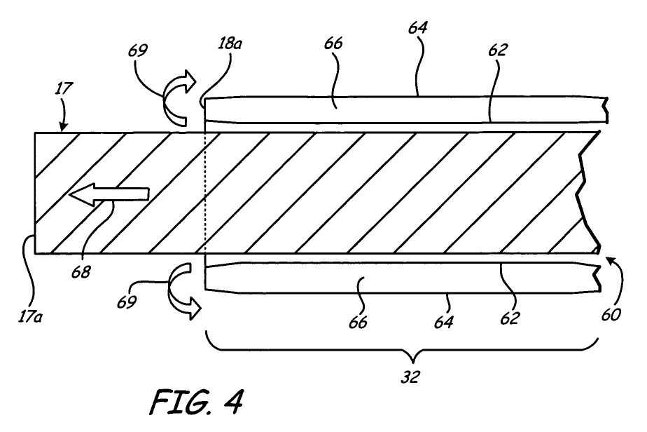

In one embodiment, the inner layer of sheath 18 is frictionally attached to the outer diameter of endoscope 17 when sheath 18 is inflated and deflated. This allows the inner layer of sheath 18 to evert out the distal end of the sheath when sheath 18 and expandable member 20 are inflated and endoscope 17 is advanced relative to expandable member 20. Inflation of sheath 18 provides an area of lubrication between the inner and outer layers of the sheath material to allow expandable member 20 to slide back and forth relative to endoscope 17, as shown in more detail in FIGS. 4 and 5.

FIG. 4 is an enlarged, cross-sectional view of the distal end 18a of everting sheath 18 when inflated. Endoscope 17 extends through an internal lumen 60 of sheath 18. Sheath 18 has a cylindrical inner layer 62 and a cylindrical outer layer 64, which together form an elongated, annular tube. At distal end 18a, inner layer 62 is attached to outer layer 64, forming a continuous material, for example. Cavity 66 between inner layer 62 and outer layer 64 is inflated by introducing a pressurized fluid (such as water) within the cavity. Inflation of the space between the inner and outer layers of sheath 18 separates elastic bands 26 and expandable member 20 from endoscope 17 and allows the endoscope to move independently from expandable member 20.

When sheath 18 and expandable member 20 are inflated and endoscope 17 is advanced relative to expandable member 20, as shown by arrow 68, the frictional engagement between inner layer 62 and the outer diameter of endoscope 17 causes the inner layer 62 to travel with endoscope 17 relative to expandable member 20 and outer layer 64. As endoscope 17 continues to advance, more of the inner layer material gets everted out of distal end 18a, as shown by arrows 69, thereby forming additional outer layer material. This eversion action increases the length of everted section 32 that extends forward beyond expandable member 20 (not shown in FIG. 4). As mentioned above, the movement of inner layer 62 relative to outer layer 64 about the fluid in cavity 66 provides an area of lubrication to allow expandable member 20 to slide back and forth relative to endoscope 17, and the inner layer 62 is able to pass freely under expandable member 20.

FIG. 5 is an expanded view of the distal end 18a of sheath 18 according to one embodiment of the present invention. When sheath 18 and expandable member 20 are inflated, fluid pressure inside the expandable member and the sheath overcomes the grip of elastic bands 26 and allows expandable member 20 to expand from a first diameter to a second, larger diameter. When elastic bands 26 are released from endoscope 17, the endoscope is free to move relative to the fixed expandable member 20. Expandable member 20 can have any number of sections. In the embodiment shown in FIG. 5, expandable member 20 has three sections separated by two elastic bands 26. When inflated, expandable member 20 engages the inner surface of colon 12. This allows the location of expandable member 20 and the outer layer 64 of sheath 18 to remain fixed relative to colon 12.

As mentioned above, expandable member 20 can be inflated and deflated either separately or with sheath 18. In the embodiment shown in FIG. 5, expandable member 20 has an internal cavity 80, which is coupled to lumen 46 for inflating expandable member 20 separately from sheath 18.

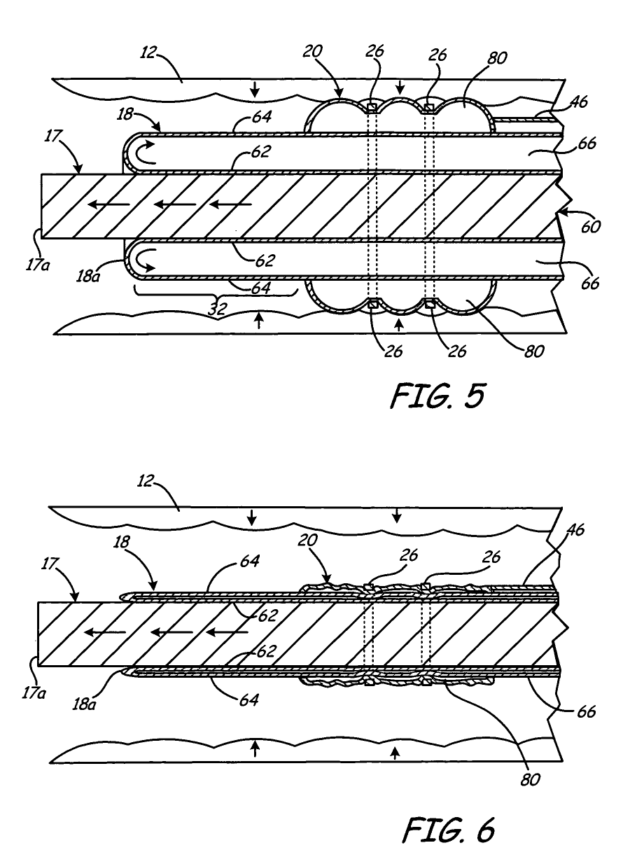

FIG. 6 cross-sectional view of the distal end 18a of sheath 18 when sheath 18 and expandable member 20 are deflated. When deflated, elastic bands 26 collapse expandable member 20 until the bands grip the outer diameter of endoscope 17 through outer layer 64 and inner layer 62 of sheath 18. In this state, endoscope 17 and sheath 18 can be advanced or withdrawn together through colon 12.

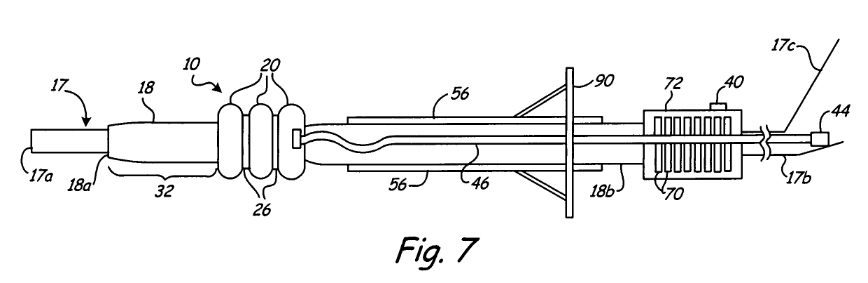

FIG. 7 is a side view of endoscope 17 and sheath 18 showing sheath 18 in a partially everted state, relative to FIG. 3. As endoscope 17 is advanced relative to inflated expandable member 20, the length of everted section 32 forward of expandable member 20 increases, as can be seen with a comparison to FIG. 3. Also, more of endoscope 17 becomes exposed beyond the distal end 18a of sheath 18.

FIG. 7 also illustrates an alternative embodiment in which extra sheath material 70 is stored in a housing 72 containing inflation valve 40. The extra sheath material can be stored in a housing separate from inflation valve 40 in a further alternative embodiment. In one embodiment, the inner layer of sheath 18 forms a continuous piece of material with the outer layer at proximal end 18b, similar to distal end 18a. However, the inner and outer layers can be disconnected from one another at proximal end 18b in an alternative embodiment of the present invention.

FIG. 7 further illustrates an abutment device 92 fastened to the ridges 56 along the outer layer of sheath 18. Abutment device 92 can be used to anchor the axial position of the outer layer of sheath 18 relative to the patient's rectum as endoscope 17 is advanced. As described with reference to FIGS. 1-1 through 1-14, the colon can be straightened by inflating expandable member 20 and then pulling back on endoscope 17 and sheath 18. If the physician desires not to withdraw endoscope 17 while straightening the colon, the physician can pull back on sheath 18 while pushing endoscope 17 forward. This may require the physician to pull back on sheath 18 with one hand while pushing forward on endoscope 17 with the other hand. Abutment device 90 can help to free one of the physician's hands while advancing the endoscope 18. As sheath 18 is being pulled backward, abutment device 90 can be slid forward along sheath 18 toward the patient's rectum. Abutment device 90 can therefore act as a stopper, which holds the position of sheath 18 relative to the colon to prevent forward movement of the sheath when pushing endoscope 17 forward. This keeps the outer diameter of sheath 18 in place and tension between the sheath and expandable member 20, thereby maintaining a straightened section of the colon.

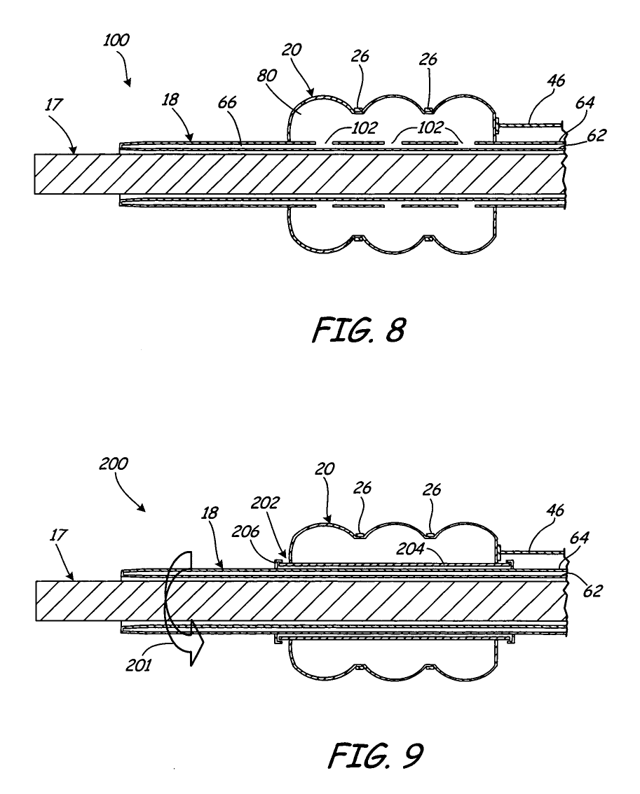

FIG. 8 is a cross-sectional view of the distal end of an endoscope apparatus 100 according to an alternative embodiment of the present invention. The same reference numerals are used in FIG. 8 as were used in the preceding figures for the same or similar elements. In this embodiment, the internal cavity 80 of expandable member 20 is coupled to the cavity 66 between the inner and outer layers 62 and 64 of sheath 18 through one or more openings 102. Inflation lumen 46 is coupled to cavity 80 of expandable member 20, but could alternatively be coupled to cavity 66 of sheath 18. When a pressurized fluid is inserted into cavity 80 through inflation lumen 46 in order to inflate expandable member 20, excess fluid enters cavity 66 through openings 102 causing sheath 18 also to inflate.

FIG. 9 is a cross-sectional view of the distal end of an endoscope apparatus 200 according to another alternative embodiment of the present invention. Again, the same reference numerals are used for the same or similar elements. In FIG. 9, endoscope apparatus 200 has an angular position that is rotatable relative to expandable member 20, as indicated by arrow 201. Expandable member 20 is mounted to the outer layer 64 of sheath 18 through a sliding interface or fitting 202. In this example, expandable member 20 has a base 204, which is mounted in a channel 206 on outer layer 64 to form the sliding interface 202. Channel 206 has an annular shape, which extends around the circumference of outer layer 64. Other methods and structures for mounting expandable member 20 to apparatus 10 that allow for relative rotational movement can be used in alternative embodiments of the present invention.

During some procedures, it may be desirable to apply a rotational torque on the endoscope 17 in order to direct the endoscope 17 around difficult turns or to fine-tune the position of the endoscope 17 when taking a biopsy or removing a polyp, for example. By making exapandable member 20 completely separate from sheath 18, endoscope 17 can rotate while expandable member 20 remains anchored to the colon in a fixed position.

A rotatable expandable member or balloon, as shown in FIG. 9 can be implemented on any elongated medical device, such as an endoscope, endoscope sheath or catheter, for example. If a sheath is used, the sheath can be everting or non-everting.

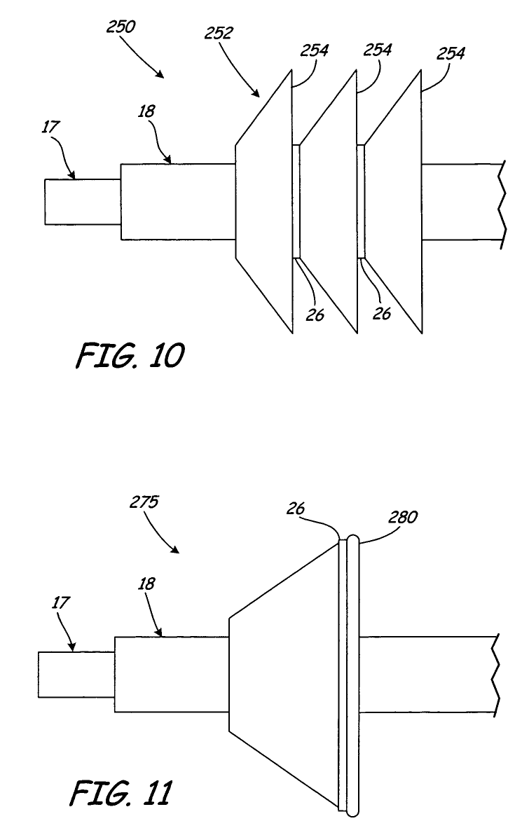

Various shapes and configurations of the expandable member can be used to improve the longitudinal traction between the colon and the expandable member without creating excessive radial force against the colon. One such configuration is shown in FIG. 10. Endoscope apparatus 250 includes an endoscope 17 and an everting sheath 18, which are similar to the embodiments discussed above. In this embodiment, sheath 18 includes an expandable member 252 having a shape that allows for the expandable member to catch or grab on to areas of the colon when withdrawn as in the action of an anchor, but would not require excessive inflation and over-expand the colon. When expanded, expandable member 252 forms three anchor fins 254, which project radially outward from sheath 18 for engaging the colon wall. Fins 254 can extend around all or part of the circumference and can have any suitable cross-sectional shape, such as triangular.

FIG. 11 illustrates a further embodiment of an endoscope apparatus 275 having an expandable member 280 attached to everting sheath 18. In this embodiment, expandable member 280 has a conical shape. Elastic band 26 biases expandable member 280 to the smaller diameter when not inflated.

In a further embodiment, the expandable member can be formed of a mechanical anchor attached to the sheath, which could be made of plastic or other suitable material. The mechanical anchor could be activated (expanded and/or contracted) by means of a wire-triggered (or other) mechanism, rather than by inflation with a fluid or gas.

With the embodiments shown in the above-described figures, an everting sheath can be preloaded on any standard or specialized endoscope. The sheath carries an expandable member, which can either be fixed to the endoscope by elastic bands (for example) when deflated, or detached from the endoscope when inflated and anchored to the colon. This allows the endoscope and expandable member to be moved either together as a single unit or independently. This device also may remove the need to invest in complicated and expensive endoscopes with special features to allow the endoscope to be advanced easily through the colon. Rather, a standard endoscope can be preloaded with an everting sheath as described above. This can significantly reduce the expense associated with colonoscopy procedures.

Also, the use of this device may allow the endoscope to be advanced to the end of the colon much faster than endoscopes of the prior art. This significantly reduces the time required for a colonoscopy procedure and reduces the duration of patient discomfort. In addition, the ability to straighten certain difficult sections of the colon allows the endoscope to be advanced with reduced pain to the patient. As a result, physicians may find that colonoscopy procedures can be performed more safely and with less sedation.

Although the present invention has been described with reference to preferred embodiments, workers skilled in the art will recognize that changes may be made in form and detail without departing from the scope of the invention.

What is claimed is:

1. A sheath for assisting movement of an endoscope within a cavity of a patient's body, the sheath comprising:
an everting shaft comprising proximal and distal regions and a lumen for receiving the endoscope; and
an expandable member along the distal region, which is expandable from a first diameter to a second, larger diameter for engaging a wall of the cavity.

2. The sheath of claim 1 wherein the first diameter of the expandable member is sized for gripping the endoscope within the lumen and the second diameter of the expandable member is sized for releasing the endoscope and engaging the wall of the cavity.

3. The sheath of claim 1 wherein:
the everting shaft comprises:
an inner layer, which circumscribes the lumen;
an outer layer, which circumscribes the inner layer and is coupled to the inner layer at a distal end of the everting shaft such that movement of the inner layer toward the distal end relative to the outer layer enables a distal portion of the inner layer to evert to the outer layer during the movement; and
the expandable member is positioned along the distal end of the everting shaft and longitudinally fixed relative to the outer layer.

4. The sheath of claim 3, wherein the expandable member comprises a first cavity, which is adapted to receive an inflation fluid, wherein insertion of the inflation fluid at a predetermined pressure expands the expandable member from the first diameter toward the second diameter.

5. The sheath of claim 4 wherein the everting shaft further comprises a collapsible, second cavity between the inner and outer layers, which is adapted to receive an inflation fluid, wherein insertion of the inflation fluid within the second cavity increases separation between the inner and outer layers.

6. The sheath of claim 5, wherein the first and second cavities are coupled to one another.

7. The sheath of claim 3 wherein the inner layer is coupled to the outer layer in a contiguous fashion at a proximal end of the everting shaft.

8. The sheath of claim 3 and further comprising a housing located along the proximal region for housing excess material of the inner layer, which allows extension of the inner layer toward the distal region relative to the outer layer.

9. The sheath of claim 3 wherein the expandable member is formed as a single, continuous piece of material with the outer layer and is defined relative to the outer layer by one or more regions of reduced material thickness.

10. The sheath of claim 3 wherein the expandable member is a separate piece of material from the outer layer and is mounted to the outer layer.

11. The sheath of claim 10 wherein and the expandable member is mounted to the everting shaft such that the everting has an angular position that is rotatable relative to the expandable member.

12. The sheath of claim 1 wherein the expandable member comprises at least one elastic element, which biases the expandable member toward the first diameter.

13. The sheath of claim 12, wherein the elastic element comprises at least one elastic band, which circumscribes the expandable member.

14. The sheath of claim 12, wherein the expandable member comprises at least one region of a first elasticity and at least one region of a second, lesser material elasticity.

15. The sheath of claim 1 and further comprising an abutment device which is connectable to an outer diameter surface of the everting shaft along the proximal region.

16. The sheath of claim 1 wherein the expandable member is actuated by a mechanical mechanism.

17. An endoscope apparatus comprising:
an everting shaft comprising inner and outer layers and a lumen defined by the inner layer;
an endoscope having a shaft extending along the lumen and engaged with the inner layer; and
an expandable member along a distal region of the outer layer, which is expandable from a first diameter to a second, larger diameter.

18. The endoscope apparatus of claim 17 wherein:
the endoscope shaft frictionally engages the inner layer such that the inner layer moves with the endoscope shaft relative to the outer layer when the expandable member has the second diameter;
the inner layer is coupled to the outer layer at a distal end of the everting shaft such that movement of the inner layer toward the distal end relative to the outer layer enables a distal portion of the inner layer to evert to the outer layer during the movement.

19. The endoscope apparatus of claim 17 wherein the everting shaft further comprises an inflation cavity between the inner and outer layers, which is adapted to receive an inflation fluid, wherein insertion of the inflation fluid within the inflation cavity increases separation between the inner and outer layers.

20. The endoscope apparatus of claim 17, wherein the expandable member comprises an inflation cavity, which is adapted to receive an inflation fluid, wherein insertion of the inflation fluid within the inflation cavity expands the expandable member from the first diameter toward the second diameter.

21. The endoscope apparatus of claim 17 wherein the expandable member grips the endoscope shaft at the first diameter and is separated from the endoscope shaft at the second diameter.

22. The sheath of claim 17 wherein and the expandable member is mounted to the everting shaft such that the everting shaft has an angular position that is rotatable relative to the expandable member when at the second diameter.

23. The endoscope apparatus of claim 17 wherein apparatus is adapted for movement within a body cavity of a patient and wherein the expandable member is sized to engage a wall of the body cavity when expanded to the second diameter.

24. The endoscope apparatus of claim 17 wherein the expandable member comprises at least one elastic element, which biases the expandable member toward the first diameter.

25. The endoscope apparatus of claim 24, wherein the at least one elastic element comprises at least one elastic band, which circumscribes the expandable member.

26. The endoscope apparatus of claim 17 wherein the expandable member is actuated by a mechanical mechanism.

27. The endoscope apparatus of claim 17 and further comprising an abutment device which is connectable to the outer layer along a proximal region of the everting shaft.

28. A method of moving an endoscope along an elongated body cavity, the method comprising:
(a) inserting an endoscope apparatus into the body cavity, the apparatus comprising an endoscope and an everting shaft, which includes inner and outer layers, a lumen through which the endoscope extends, and an expandable member;
(b) advancing the apparatus to a portion of the body cavity at which position the endoscope does not easily progress with the application of inserting force upon the endoscope with the expandable member contracted to a first diameter;
(c) inflating the sheath and expanding the expandable member from the first diameter toward a second, larger diameter at which the expandable anchors to a wall of the body cavity;
(d) applying a withdrawing force on the outer layer when the expandable member is anchored to the wall of the body cavity to thereby reduce curvature and stretch of the portion of the body cavity; and
(e) advancing the endoscope after or during step (d) relative to the expandable member such that engagement between the endoscope and the inner layer causes a distal portion of the inner layer to evert to the outer layer during advancement.

29. The method of claim 28 wherein the expandable member grips the endoscope at the first diameter and is separated from the endoscope at the second diameter.

30. The method of claim 29 and further comprising:
(f) contracting the expandable member to the first diameter, after step (e), to release the wall of the body cavity and re-grip the endoscope; and
(g) further advancing the endoscope and everting shaft together after step (f), with the expandable member gripping the endoscope.

31. The method of claim 30 and further comprising, after step (g):
(h) expanding the expandable member from the first diameter; and
(i) withdrawing the endoscope relative to the expandable member when the expandable member is expanded;
(j) contracting the expandable member to the first diameter, after step (i), to re-grip the endoscope at a more favourable position at the distal end of the endoscope; and
(k) further advancing the endoscope and the everting shaft together after step (j), with the expandable member gripping the endoscope.

32. The method of claim 28 and further comprising rotating an angular position of the endoscope and everting shaft relative to the expandable member when the expandable member is expanded to the second diameter and anchored to the wall of the body cavity.

33. A medical device for insertion into a body cavity, the device comprising:
an elongated shaft; and
an expandable member, which is expandable from a first diameter to a second, larger diameter and is mounted to the elongated shaft such that the elongated shaft has an angular position that is rotatable relative to the expandable member.

34. The medical device of claim 33 wherein the elongated shaft comprises a slot and the expandable member is mounted within the slot so as to allow angular rotation within the slot.

35. The medical device of claim 33 wherein:
the elongated shaft comprises an everting sheath comprising proximal and distal regions and a lumen for receiving an endoscope; and
the expandable member is mounted to an outer layer of the everting sheath and is expandable from a first diameter to a second, larger diameter.

36. The medical device of claim 35 wherein the first diameter of the expandable member is sized to grip the endoscope within the lumen and the second diameter is sized to grip the wall of the body cavity.

37. The medical device of claim 36 wherein the expandable member comprises at least one elastic element, which biases the expandable member toward the first diameter, and wherein expansion of a space between the inner and outer layers of the everting sheath expands the elastic element from the first diameter.
